(19) United States
(12) Patent Application Publication
(10) Pub. No.: US 2021/0290244 A1
(43) Pub. Date: Sep. 23, 2021

(54) CARTRIDGE, CLIP SYSTEM, AND METHOD OF MOUNTING CLIP

(71) Applicant: OLYMPUS CORPORATION, Tokyo (JP)

(72) Inventors: Yasuyuki FUJIMOTO, Hino-shi (JP); Kensuke UESAKA, Hino-shi (JP); Ryu YORITA, Hachioji-shi (JP); Shinya ANSAI, Tachikawa-shi (JP)

(73) Assignee: OLYMPUS CORPORATION, Tokyo (JP)

(21) Appl. No.: 17/342,219

(22) Filed: Jun. 8, 2021

Related U.S. Application Data
(63) Continuation of application No. PCT/JP2018/045453, filed on Dec. 11, 2018.

(51) Int. Cl. A61B 17/128

(52) U.S. Cl. CPC A61B 17/1285; A61B 2017/00367

(57) ABSTRACT

A cartridge accommodates a clip unit including an arm member and a tubular housing, and is configured so that the clip unit is able to be mounted in an applicator having a sheath and an operation wire. The cartridge includes a main body in which an insertion hole into which the sheath is inserted is formed and a pusher configured to be movable with respect to the main body. The pusher is configured to come into contact with an engaging member provided in the sheath by moving the sheath with respect to the main body in a state in which the sheath is inserted into the insertion hole. The pusher is configured to push the engaging member against the housing, and to engage the sheath with the housing by engaging the engaging member with both the housing and the sheath.

The present invention relates to a clip cartridge for ligating body tissues, a clip system, and a method of mounting a clip. This application is a continuation application based on International Patent Application No. PCT/JP 2018/045453 filed on Dec. 11, 2018, and the content of the PCT international application is incorporated herein by reference.

### BACKGROUND

Conventionally, a clip device for ligating a tissue to be treated with a clip to close an opening formed in a living tissue, to perform a hemostatic treatment, or the like is known. The clip device includes an operation part, a sheath, and an operation wire which is inserted into the sheath and connected to the operation part. In the clip device, the sheath is inserted into an endoscopic channel and inserted into a body via the channel.

For example, United States Patent Application Publication No. 2012/0109160 discloses a clip device in which a pair of jaws are rotatably supported by a housing. In the pair of jaws, a driver to which a distal head of a drive wire is connected with proximal end portions of the pair of jaws by a rack-pinion structure, and the pair of jaws rotate with respect to the housing to be opened and closed as the drive wire advances and retracts.

The clip device for placing a clip in the body is inserted into the body in a state that the clip is connected to a distal end of the sheath. Arms (the pair of jaws) of the clip are opened by operating the drive wire to bring them into contact with the tissue, and then the tissue is ligated by closing the clip. After that, the connection between the clip and the drive wire is released, and the clip is indwelled in the body. Therefore, before the clip is indwelled inside the body, the clip and the distal end of the endoscopic channel are connected, and after the clip is indwelled, it is necessary to disconnect the clip from the distal end of the endoscopic channel.

In the clip device of United States Patent Application Publication No. 2012/0109160, a connection block is provided inside a tubular connector, the connection block protrudes from the tubular connector and is press-fitted into the housing by operating the drive wire, and the housing and the tubular connector are fixed to each other via the connection block.

### SUMMARY

A cartridge according to a first aspect is a cartridge configured to accommodate a clip unit that includes an arm member and a tubular housing into which the arm member is inserted, the cartridge further configured so that the clip unit is able to be mounted in an applicator, the applicator having a sheath that is engageable with the housing and an operation wire which is engageable with the arm member. The cartridge includes a main body and a pusher. The main body has an insertion hole that is configured to receive the sheath. The pusher is configured to be movable with respect to the main body, and the pusher is able to come into contact with an engaging member by moving the sheath with respect to the main body in a state in which the sheath is inserted into the insertion hole, the pusher configured to push the engaging member against the housing while in contact with the engaging member, and engages the sheath with the housing by engaging the engaging member with both the housing and the sheath. The engaging member being provided in the sheath.

According to a second aspect, in the cartridge according to the first aspect, the insertion hole may have an elongated shape into which the sheath is able to be mounted, and the pusher may be configured to be movable in a direction crossing the longitudinal axis of the insertion hole.

According to a third aspect, in the cartridge according to the second aspect, the pusher may be configured to protrude into the insertion hole.

According to a fourth aspect, in the cartridge according to the third aspect, the pusher may be configured to come into contact with the engaging member, the engaging member being accommodated in the sheath by passing through an opening formed in an outer circumferential surface of the sheath and protruding into the insertion hole.

According to a fifth aspect, in the cartridge according to the fourth aspect, the pusher may have an inclined surface that is inclined with respect to the longitudinal axis of the insertion hole, and the inclined surface of the pusher may be configured to be able to come into contact with the engaging member.

A clip system according to a sixth aspect includes a clip unit including an arm member and a housing having a tubular shape, the arm member being inserted into the housing, an applicator having a sheath that is engageable with the housing, an operation wire which is engageable with the arm member, and an engaging member that is provided in the sheath and configured to be engaged with both the sheath and the housing and configured to move from the sheath toward the housing.

According to a seventh aspect, the clip system according to the sixth aspect may further include a pusher configured to move the engaging member from the sheath toward the housing, the pusher moving with respect to the sheath, the engaging member engaging with both the sheath and the housing as a consequence of the movement by the pusher.

According to an eighth aspect, in the clip system according to the sixth aspect, the pusher may be configured to come into contact with the engaging member by moving with respect to the sheath and to push the engaging member against the housing, and the pusher may be configured to press-fit the engaging member into the housing.

According to a ninth aspect, in the clip system according to the seventh aspect, the pusher may be configured to come into contact with the engaging member by moving with respect to the sheath and to push the engaging member against the housing, and the pusher may be configured to allow engaging member to protrude from the sheath.

According to a tenth aspect, in the clip system according to the seventh aspect, an opening may be formed in an outer circumferential surface of the sheath, the pusher may be configured to come into contact with the engaging member by moving with respect to the sheath and to push the engaging member against the housing; and the pusher may be configured to come into contact with the engaging member, the engaging member accommodated in the sheath by passing through the opening of the sheath and protruding into the insertion hole.

According to an eleventh aspect, in the clip system according to the seventh aspect, a cartridge including a main body having an accommodation part into which the clip unit is accommodated and an insertion hole into which the sheath is inserted may be included, the pusher may be configured to come into contact with the engaging member by moving with respect to the sheath and to push the engaging member against the housing, and the pusher may be provided on the cartridge and may be movable with respect to the main body.

According to a twelfth aspect, in the clip system according to the seventh aspect, the engaging member may be accommodated in the sheath, the pusher may be configured to come into contact with the engaging member by moving with respect to the sheath and to push the engaging member against the housing, the pusher may be accommodated in the sheath, and the pusher may be configured to be able to come into contact with the engaging member by advancing and retracting with respect to the operation wire.

According to a thirteenth aspect, in the clip system according to the seventh aspect, the pusher may include the sheath and an outer sheath into which the housing is insertable, the engaging member may be provided on the outer sheath, and the engaging member may be able to engage the housing with the sheath due to friction between an outer circumferential surface of the housing and an outer circumferential surface of the sheath as a consequence of the outer sheath moving the engaging member by toward the housing.

An engaging method according to a fourteenth aspect is a method of approaching a sheath to a cartridge that accommodates an arm member and a housing into which the arm member is inserted; moving an engaging member at least partially accommodated in the sheath from the sheath toward the housing in a state in which the sheath is close to (e.g., within a predetermined distance from) the housing; and moving the engaging member so as to engage the engaging member with both the housing and the sheath.

According to a fifteenth aspect, in the engaging method according to the fourteenth aspect, the engaging member may be inserted into the housing by moving the engaging member from the sheath toward the housing.

According to a sixteenth aspect, in the engaging method according to the fourteenth aspect, the arm member may be closed by moving the arm member from the housing toward the sheath.

According to a seventeenth aspect, in the engaging method according to the fourteenth aspect, the engaging member may be moved from the sheath toward the housing by a pusher configured to move independently of the operation wire.

According to a eighteenth aspect, in the engaging method according to the seventeenth aspect, when the engaging member is moved, the pusher may be brought into contact with the engaging member by moving the pusher with respect to the cartridge in a state in which the sheath is inserted into the insertion hole provided in the cartridge, and the engaging member may be pushed against the housing by further moving the pusher from a state in which the pusher is in contact with the engaging member.

### BRIEF DESCRIPTION OF THE DRAWINGS

FIG. 1 is an overall view showing a cartridge and a clip system according to a first embodiment.

FIG. 2 is an overall view showing the cartridge and the clip system according to the first embodiment.

FIG. 3 is an overall view showing the cartridge and the clip system according to the first embodiment.

FIG. 4 is a perspective view showing an engaging member of the first embodiment.

FIG. 5 is a schematic view showing a usage mode of the cartridge and the clip system according to the first embodiment.

FIG. 6 is a schematic view showing the usage mode of the cartridge and the clip system according to the first embodiment.

FIG. 7 is a schematic view showing the usage mode of the cartridge and the clip system according to the first embodiment.

FIG. 8 is a schematic view showing the usage mode of the cartridge and the clip system according to the first embodiment.

FIG. 9 is a flowchart showing an engagement method according to the first embodiment.

FIG. 10 is an overall view showing a modified example of the cartridge and clip system according to the first embodiment.

FIG. 11 is an overall view showing the modified example of the cartridge and clip system according to the first embodiment.

FIG. 12 is a schematic view showing a cartridge and a clip system according to a second embodiment.

FIG. 13 is a schematic view showing a cartridge and a clip system according to the second embodiment.

FIG. 14 is a schematic view showing a cartridge and a clip system according to a third embodiment.

FIG. 15 is a schematic view showing a cartridge and a clip system according to the third embodiment.

### DETAILED DESCRIPTION OF EXEMPLARY EMBODIMENTS

### First Embodiment

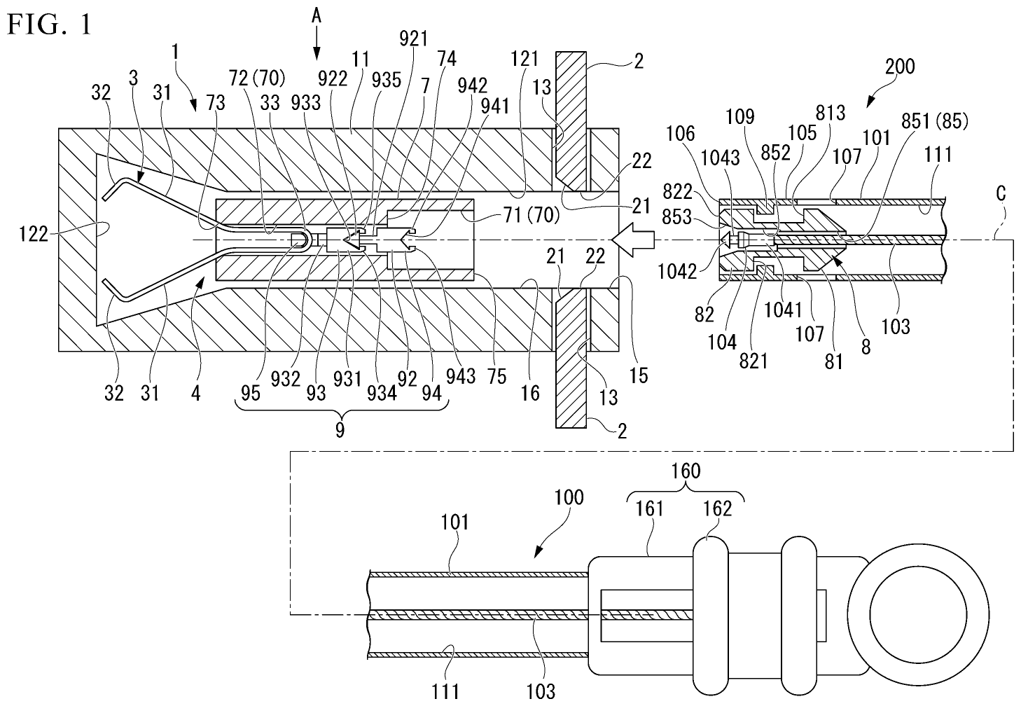

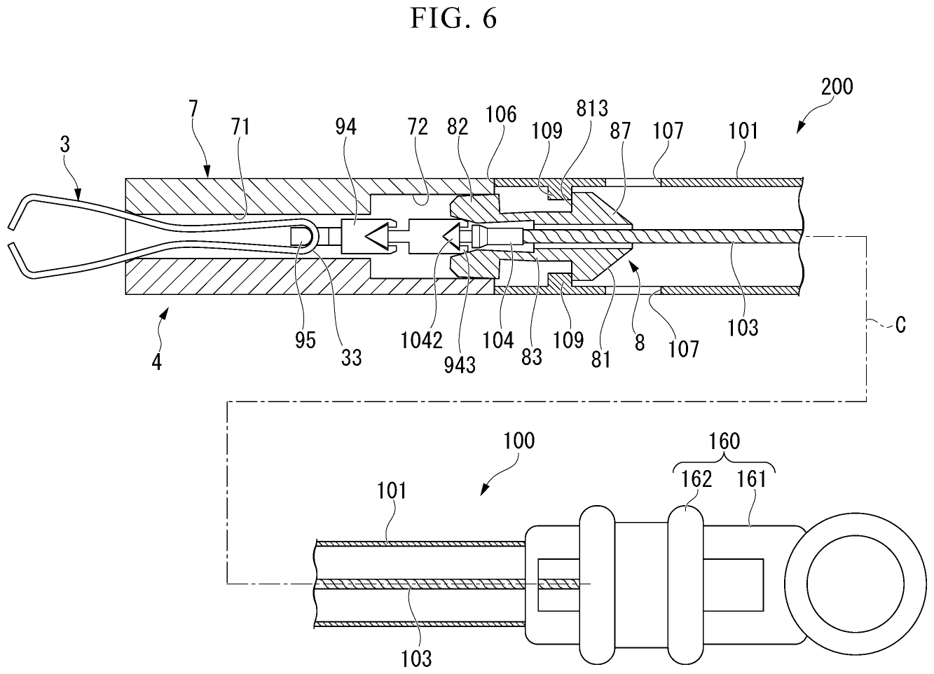

A cartridge and a clip system according to a first embodiment will be described with reference to FIGS. 1 to 8. FIGS. 1 to 3 are overall views showing the cartridge 1 and the clip system 200 according to the present embodiment. FIGS. 1 to 3 are views showing an aspect of each of processes when the cartridge 1 is used. As shown in FIGS. 1 to 3, the clip system 200 includes a clip unit 4, an applicator 100, a cartridge 1, and a pusher 2.

The clip system 200 is a system in which the applicator 100 in which the clip unit 4 is mounted is inserted into a known endoscope insertion part, and the clip unit 4 is capable of being indwelled in a body. The clip system 200 is configured so that the clip unit 4 is capable of being mounted in the applicator 100 a plurality of times by using the cartridge 1.

In the following description, a center line in a longitudinal direction in a state in which the applicator 100 extends linearly is referred to as a longitudinal axis C. The operation part 160 side of the applicator 100 is referred to as a proximal side, and the side opposite to the proximal side in a direction of the longitudinal axis C and in which the clip unit 4 is provided is referred to as a distal side.

First, the clip unit 4 will be described.

The clip unit 4 includes a housing 7 and an arm member 3. As shown in FIG. 1, the housing 7 has a tubular shape through which a hole 70 passes in the direction of the longitudinal axis C. The hole 70 of the housing 7 is a circular hole. The hole 70 is formed to communicate a first insertion hole 71 (an insertion hole) formed on the proximal side and a second insertion hole 72 formed on the distal side.

The first insertion hole 71 is a hole formed in a narrow elongated shape and has a larger opening diameter than the second insertion hole 72. The first insertion hole 71 has an opening diameter in which a sheath that will be described later is capable of being disposed. A step is formed at a boundary portion between the first insertion hole 71 and the second insertion hole 72, and a distal wall 74 which extends outward from a proximal end of the second insertion hole 72 in a direction orthogonal to the longitudinal axis C is formed at a distal end of the first insertion hole 71.

The second insertion hole 72 has an opening size into which the arm member 3 is capable of being inserted. A distal end portion of the second insertion hole 72 has a tapered surface 73 which slightly expands toward a distal end surface of the housing 7.

The housing 7 is manufactured using, for example, a metal material such as stainless steel, a titanium alloy (Ti-6AL-4V, or the like), or a cobalt-chromium alloy, or a high-strength resin material having appropriate elasticity such as polyphthalamide (PPA) or polyamide (PA).

The arm member 3 has one or more arms 31. In the present embodiment, the arm member 3 includes a pair of arms 31 (a first arm and a second arm) and a connecting part 33 on the proximal side of each of the arms 31. The arm member 3 is made of, for example, a thin and elongated plate made of a metal such as stainless steel, a cobalt-chromium alloy, or titanium. In the pair of arms 31, the U-shaped connecting part 33 is formed at a middle portion of the thin and elongated plate by bending in a thickness direction, and both end portions of the plate serve as the pair of arms 31. Claws 32 bent toward each other are formed at end portions of the arms 31 on the side opposite to the connecting part 33. The pair of arms 31 have a bending characteristic in such that arms 31 are gradually away from each other from the connecting part 33 side toward the claw 32 side.

The clip unit 4 further includes a connection member 9. The connection member 9 is a member which connects the arm member 3 to an operation wire 103. The connection member 9 is provided at a proximal end portion of the arm member 3 in the housing 7. The connection member 9 includes a first connection body 92 and a second connection body 93. The first connection body 92 and the second connection body 93 are arranged in the direction of the longitudinal axis C. The connection member 9 is configured such that the first connection body 92 and the second connection body 93 are engaged with each other.

The first connection body 92 is located at a proximal part of the connection member 9 and engages with the operation wire 103. A wire engaging part 94 is provided at a proximal end part of the first connection body 92. The wire engaging part 94 includes an engaging hole 941, a pair of engaging arms 942, and a locking part 943. The engaging hole 941 is a hole which opens in a proximal end surface of the first connection body 92. The engaging hole 941 has a size into which a connector 104 of the operation wire 103 which will be described later is insertable. A pair of engaging arms 942 are provided at a proximal edge part of the engaging hole 941 to protrude toward a proximal direction. Each of the pair of engaging arms 942 is configured to be able to bend outward in a radial direction of the first connection body 92. A locking part 943 is provided at each of proximal end portions of the pair of engaging arms 942. The locking part 943 is provided at a position at which it faces the engaging hole 941 and covers the opening.

A distal part of the first connection body 92 includes a cylindrical part 921 which extends distally in the direction of the longitudinal axis C and a conical part 922. The conical part 922 is provided at a distal end of the cylindrical part 921, and a distal end thereof is disposed at a distal end of the first connection body 92. The diameter of a proximal part of the conical part 922 is larger than the diameter of the cylindrical part 921.

The second connection body 93 is located further on the distal side than the first connection body 92 and engages with the arm member 3. The second connection body 93 includes a connection part 931 which forms a proximal region and a hook 932 which forms a distal region. The connection part 931 includes an engaging hole 933, a pair of engaging arms 934, and a locking part 935 at a proximal end portion thereof. The engaging holes 933, the pair of engaging arms 934, and the locking part 935 have the same configuration as the wire engaging part 94 of the first connection body 92.

The conical part 922 of the first connection body 92 is inserted into the engaging hole 933 of the second connection body 93, the conical part 922 is engaged with the pair of engaging arms 934 and the locking part 935 of the second connection body 93, and thus the first connection body 92 and the second connection body 93 are connected to each other.

The hook 932 is a hook having substantially an L shape when seen in a direction of an arrow A in FIG. 1. The hook 932 is provided to protrude further to the distal side than the connection part 931.

The arms 31 of the arm member 3 and the connection member 9 are locked in the first insertion hole 71 of the housing 7. Specifically, an L-shaped end part of the hook 932 of the second connection body 93 is disposed inside a curved portion of the connecting part 33 of the arm member 3 and is inserted into the first insertion hole 71 in this state. With such a configuration, in the first insertion hole 71, the arm member 3 and the connection member 9 are engaged with each other without the hook 932 being disengaged from the connecting part 33 in the direction of the longitudinal axis C and the radial direction of the housing 7.

The first insertion hole 71 has a size which allows the locked arm member 3 and the connection member 9 to be advanceable and retractable in the first insertion hole 71 in the direction of the longitudinal axis C. The arm member 3 and the connection member 9 is capable of advancing and retracting in the first insertion hole 71 as the operation wire 103 connected to the first connection body 92 advances and retracts. The details will be described later. The arm member 3 is configured so that the pair of arms 31 is capable of being opened and closed according to a relative position of the arm member 3 with respect to the first insertion hole 71 in the direction of the longitudinal axis C.

Next, the applicator 100 will be described. The applicator 100 includes a sheath 101, the operation wire 103, an engaging member 8, and an operation part 160.

The operation part 160 includes an operation main body 161 and a slider 162. The slider 162 is provided to be slidable with respect to the operation main body 161 in the direction of the longitudinal axis C.

The sheath 101 is a flexible and long tubular member. A conduit 111 is formed along the longitudinal axis C over the entire length of the sheath 101. The sheath 101 is, for example, a coil sheath formed by winding a wire made of stainless steel such as SUS301 tightly around the longitudinal axis C. A proximal end of the sheath 101 is connected to the operation main body 161 of the operation part 160. The conduit 111 opens to a distal end 106 of the sheath 101.

A convex part 109 is formed at a distal end part 105 of the sheath 101. The convex part 109 protrudes radially inward from an inner circumferential surface of the conduit 111. The convex part 109 protrudes in an annular shape from the inner circumferential surface of the entire circumference of the conduit 111. An opening of the conduit 111 is narrowed at a position of the convex part 109, but the conduit 111 is secured to such an extent that the operation wire 103 and an intermediate portion 83 of the engaging member 8 which will be described later are capable of advancing and retracting.

A through hole (an opening part) 107 which communicates with the inside and outside of the sheath 101 is formed at the distal end part 105 of the sheath 101. The through hole 107 is located further on the proximal side than the convex part 109. A pair of through holes 107 are formed at positions facing each other on the circumference of the sheath 101. The through hole 107 has an opening into which the pusher 2 is capable of being be inserted.

The operation wire 103 is inserted through the sheath 101. A proximal end of the operation wire 103 is fixed to the slider 162 of the operation part 160, and the connector 104 is fixed to a distal end of the operation wire 103. The operation wire 103 is made of a single metal wire or a metal stranded wire.

The connector 104 is a member which connects the arm member 3 with the operation wire 103. The connector 104 includes a connection end 1041, an engaged part 1042, and a recessed part 1043. The connection end 1041 has a tubular shape. The distal end of the operation wire 103 is inserted into the connection end 1041 and the connection end 1041 is fixed to the operation wire 103. The engaged part 1042 is provided at a distal end of the connector 104. The engaged part 1042 has a conical shape. The engaged part 1042 protrudes in the direction of the longitudinal axis C, and has a pointed end disposed on the distal side thereof. The recessed part 1043 is located between a proximal end of the engaged part 1042 and the connection end 1041. The recessed part 1043 has a cylindrical shape in which an outer diameter is smaller than that of the engaged part 1042 and the connection end 1041.

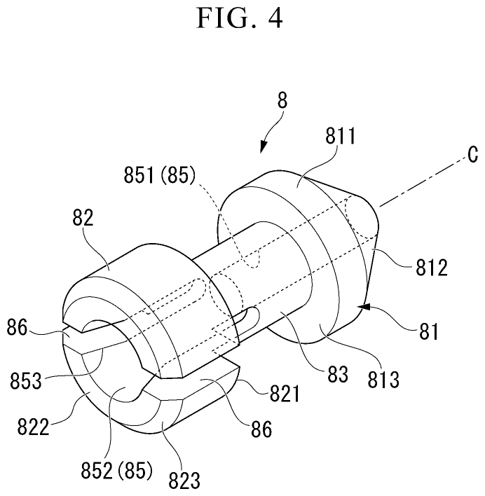

The engaging member 8 is mounted on a distal end portion of the operation wire 103. The engaging member 8 is provided to be protrudable and retractable from a distal end of the sheath 101. FIG. 4 shows a perspective view of the engaging member 8. As shown in FIGS. 1 to 4, the engaging member 8 includes a proximal end portion 81, the intermediate portion 83, and a distal end portion 82 in the direction of the longitudinal axis C, and has an insertion through hole 85 which extends in the direction of the longitudinal axis C over the entire length of the engaging member 8.

The proximal end portion 81 includes an annular portion 811 and a tapered portion 812. As shown in FIG. 1, the annular portion 811 has an annular shape having a diameter smaller than an inner diameter of the conduit 111 of the sheath 101. The tapered portion 812 is located on the proximal side further than the annular portion 811 and has a substantially conical shape in which a diameter is gradually reduced from the outer circumferential end portion of the annular portion 811 on the proximal side toward the proximal direction.

The distal end portion 82 has a substantially cylindrical shape. A chamfered slope 823 is provided at a boundary portion between an outer circumferential surface of the distal end portion 82 and a distal end surface 822. The intermediate portion 83 is located between the proximal end portion 81 and the distal end portion 82. The intermediate portion 83 has a substantially cylindrical shape having a diameter smaller than an outer diameter of the annular portion 811 of the proximal end portion 81 and the distal end portion 82. Therefore, in the engaging member 8, the intermediate portion 83 between the proximal end portion 81 and the distal end portion 82 is a recessed part.

A first contact wall 813 which extends in a direction crossing the longitudinal axis C is provided between a proximal end of the intermediate portion 83 and a distal end of an outer circumferential portion of the annular portion 811. A second contact wall 821 which extends in a direction crossing the longitudinal axis C is provided between a distal end of the intermediate portion 83 and a proximal end of an outer circumferential portion of the distal end portion 82.

A pair of slits 86 which extend from the distal end surface 822 of the engaging member 8 in the direction of the longitudinal axis C are provided. The pair of slits 86 are formed at positions facing each other with a central axis of the cylindrical distal end portion 82 interposed therebetween when the engaging member 8 is seen in the direction of the longitudinal axis C. That is, the pair of slits 86 are formed at two positions on the diameter of the distal end portion 82. The pair of slits 86 are formed to extend in the direction of the longitudinal axis C from the distal end surface 822 to a substantially center portion of the engaging member 8 in the direction of the longitudinal axis C. Therefore, the distal end portion 82 is connected to the intermediate portion 83 in a state in which it is divided into semicircles by the pair of slits 86. The pair of slits 86 extend to the intermediate portion 83.

As shown in FIG. 1, the insertion through hole 85 has different opening dimensions with the substantially center portion of the engaging member 8 in the direction of the longitudinal axis C as a boundary. In the opening dimension of the insertion through hole 85, the opening dimension of a second insertion through hole 852 located at a distal part is larger than the opening dimension of a first insertion through hole 851 located at a proximal part. The second insertion through hole 852 has an inner tapered surface 853 of which a diameter of an opening expands toward the distal end surface 822. The pair of slits 86 extend from the distal end surface 822 to a proximal end of the second insertion through hole 852. As shown in FIG. 1, the distal region of the intermediate portion 83 is thinner than the distal end portion 82 and the proximal region of the intermediate portion 83.

As a result, when an external force is applied to the distal end portions 82 in a direction orthogonal to a line (a diameter) which connects the pair of slits 86 when seen in the direction of the longitudinal axis C, the engaging member 8 bends in the distal region of the intermediate portion 83, and the divided distal end portions 82 approach each other. On the other hand, in a natural state in which the external force is not applied to the engaging member 8, the divided distal end portions 82 are maintained at separate positions. When a force is applied to the distal end portions 82 from the outside at positions separated from the slits 86 in a circumferential direction, not limited to the direction orthogonal to the line (the diameter) which connects the pair of slits 86, the distal side of the engaging member 8 bends, and the divided distal end portions 82 approach each other.

As shown in FIG. 1, in the engaging member 8, the operation wire 103 is inserted into the insertion through hole 85 to be advanceable and retractable. A distal part of the operation wire 103 passes through the first insertion through hole 851, and the connector 104 is inserted into the second insertion through hole 852. External dimensions of a distal part of the connection end 1041 and the engaged part 1042 are larger than an opening dimension of the first insertion through hole 851. Therefore, the connector 104 comes into contact with the distal end of the first insertion through hole 851 when the connector 104 retracts with respect to the engaging member 8. Therefore, the operation wire 103 does not come off from the engaging member 8 to the proximal side.

As shown in FIG. 1, the engaging member 8 is disposed inside the distal end part 105 of the sheath 101. The intermediate portion 83 of the engaging member 8 is inserted inside the annular convex part 109 of the sheath 101. The engaging member 8 is provided to be advanceable and retractable in the distal end part 105 of the sheath 101. An inner opening diameter of the convex part 109 is larger than an outer diameter of the intermediate portion 83 and is smaller than an outer diameter of the proximal end portion 81 and the distal end portion 82. The engaging member 8 is configured to be advanceable and retractable from a position at which the first contact wall 813 of the proximal end portion 81 comes into contact with the convex part 109 to a position at which the second contact wall 821 of the distal end portion 82 comes into contact with the convex part 109. When the engaging member 8 advances, the first contact wall 813 of the proximal end portion 81 comes into contact with the convex part 109, and further advance is restricted. When the engaging member 8 retracts, the second contact wall 821 of the distal end portion 82 comes into contact with the convex part 109, and further retraction is restricted. Therefore, an amount of advance and retraction of the engaging member 8 with respect to the sheath 101 is defined by the distance between the first contact wall 813 and the second contact wall 821.

The length of the distal end portion 82 in the direction of the longitudinal axis C is slightly shorter than the length from the distal end 106 of the sheath 101 to the distal end of the convex part 109. Therefore, as shown in FIG. 1, when the position of the engaging member 8 with respect to the sheath 101 is in the most retracted position, the engaging member 8 is accommodated inside the sheath 101 over the entire length thereof. When the engaging member 8 is at a retracted position, the proximal end portion 81 is disposed at a position of the through hole 107 of the sheath 101 in the direction of the longitudinal axis C.

The pusher 2 is configured to be movable with respect to the sheath 101 by coming into contact with the engaging member 8 and pressing the engaging member 8. In the present embodiment, the pusher 2 is provided in the cartridge 1.

The cartridge 1 accommodates the clip unit 4 before use, and is configured to be able to mount the clip unit 4 in the applicator 100. The cartridge 1 includes a main body 11, and the pusher 2 is provided in the main body 11.

As shown in FIG. 1, the main body 11 has an accommodation part 12 in which the clip unit 4 is accommodated. The accommodation part 12 is a hollow part formed according to an exterior of the clip unit 4. The accommodation part 12 has a first accommodation part 121 (an insertion hole) which accommodates the housing 7 of the clip unit 4, and a second accommodation part 122 which accommodates the distal region of the arm member 3 which has protruded from the housing 7.

The first accommodation part 121 has an elongated shape. An insertion hole (an opening) 15 is formed in the first accommodation part 121. The insertion hole 15 opens at a proximal end of the main body 11 and allows the sheath 101 to be inserted in the direction of the longitudinal axis C. The first accommodation part 121 is configured so that the sheath 101 is insertable in the direction of the longitudinal axis C (an axis direction). The first accommodation part 121 is longer than the length of the housing 7 in the direction of the longitudinal axis C.

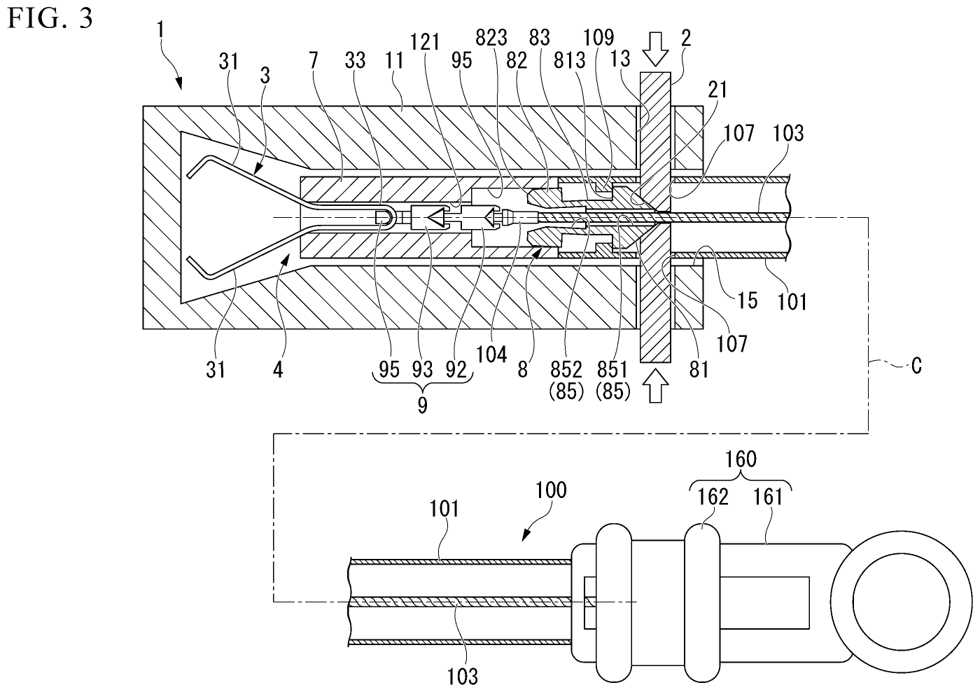

The pusher 2 is provided at a proximal end portion of the main body 11. A slide hole 13 is formed in the main body 11. The slide hole 13 communicates a proximal part of the first accommodation part 121 with an outer surface of the main body 11. The slide hole 13 is a hole which extends in a direction orthogonal to the direction of the longitudinal axis C. The pusher 2 is an elongated rod-shaped member, and is inserted into the slide hole 13. As shown in FIGS. 1 and 3, the pusher 2 is configured to be movable in a direction crossing the longitudinal axis C of the first accommodation part 121. A biasing member (not shown) is provided at the pusher 2. In the natural state in which an external force is not applied to the pusher 2, the pusher 2 is held in a state that the pusher 2 does not protrude into the first accommodation part 121 as shown in FIG. 1.

The pusher 2 has an inclined surface 21 which is inclined with respect to the longitudinal axis C. The inclined surface 21 has a shape in which a corner portion on the distal side in an inner end surface 22 which faces the first accommodation part 121 side of the pusher 2 is chamfered.

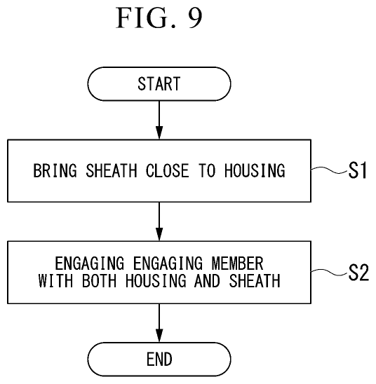

Next, an operation of the cartridge 1 and the clip system 200 and an engagement method thereof will be described. FIG. 9 is a flowchart showing an engagement method according to the present embodiment.

The clip unit 4 is accommodated in advance in the accommodation part 12 of the cartridge 1 in a state in which the arm 31 in the distal region of the arm member 3 protrudes from the housing 7 toward the distal side. In the clip unit 4 accommodated in the accommodation part 12, the housing 7 is accommodated in the distal region of the first accommodation part 121. A space into which the sheath 101 is inserted is formed in the first accommodation part 121 on the proximal side of the housing 7.

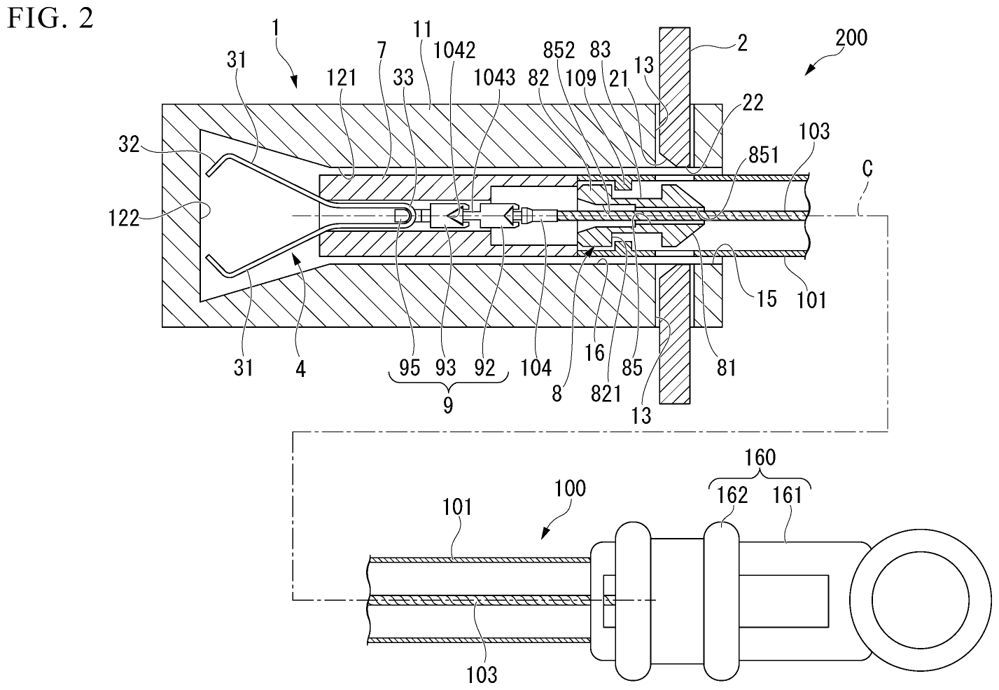

The cartridge 1 is brought close to (e.g., within a predetermined distance from) the distal end 106 of the sheath 101, and the distal end 106 is inserted into the insertion hole 15 of the first accommodation part 121 of the cartridge 1 (a first step S1). The sheath 101 is advanced to the cartridge 1 side so that the distal end 106 of the sheath 101 comes into contact with the proximal end of the housing 7, as shown in FIG. 2.

Subsequently, an operator operates the slider 162 to advance the operation wire 103 and to push the engaged part 1042 toward the engaging hole 941 side. As a result, a pointed end of the engaged part 1042 enters the engaging hole 941 from between the pair of engaging arms 942 and bends in a direction in which the pair of engaging arms 942 are separated, and the engaged part 1042 is inserted into the engaging hole 941. Since the engaging hole 941 of the wire engaging part 94 has a shape similar to a shape of the engaged part 1042, the engaged part 1042 is disposed in a state in which it is close to (e.g., within a predetermined distance from) the engaging hole 941. When the engaged part 1042 is inserted into the engaging hole 941, the pair of engaging arms 942 return to the position of the natural state, and the locking part 943 comes into contact with a proximal end surface of the engaged part 1042. As a result, the engaged part 1042 is engaged in the engaging hole 941, and the distal end of the operation wire 103 is engaged with the connection member 9.

Next, the operator pushes the pusher 2 to the first accommodation part 121 side. The pusher 2 is movable with respect to the sheath 101. The pusher 2 engages the engaging member 8 with both the sheath 101 and the housing by coming into contact with the engaging member 8 due to the movement with respect to the sheath 101 and pushing the engaging member 8 against the housing (a second step S2).

Specifically, in a state in which a proximal end 75 of the housing 7 and the distal end 106 of the sheath 101 are in contact with each other in the first accommodation part 121, positions of the through hole 107 and the slide hole 13 in the direction of the longitudinal axis C coincide with each other. In this state, when the operator pushes the pusher 2 toward the first accommodation part 121 side, the inner end surface 22 and the inclined surface 21 of the pusher 2 protrude from the slide hole 13 into the first accommodation part 121.

The pusher 2 which protrudes into the first accommodation part 121 passes through the through hole 107 of the sheath 101 and comes into contact with the proximal end portion 81 of the engaging member 8. When the operator continuously pushes the pusher 2 further, as shown in FIG. 3, the inclined surface 21 of the pusher 2 is pushed while being in contact with the tapered portion 812 of the proximal end portion 81. As a result, the engaging member 8 advances toward the distal side.

When the slope 823 of the distal end portion 82 of the engaging member 8 is pushed while being in contact with the first insertion hole 71 at the proximal end of the housing 7, the distal end portion 82 bends and enters the first insertion hole 71. When the pusher 2 is pushed close to (e.g., within a predetermined distance from) the operation wire 103, the first contact wall 813 of the proximal end portion 81 of the engaging member 8 comes into contact with the convex part 109 of the sheath 101, and substantially the entire distal end portion 82 is inserted into the first insertion hole 71. The distal end portion 82 is frictionally engaged with the first insertion hole 71. As a result, the housing 7 and the sheath 101 are in connect with each other via the engaging member 8. At this time, an opening of the second insertion through hole 852 is narrowed, but a space in which the operation wire 103 is capable of advancing and retracting is secured.

When the pusher 2 is pushed to the innermost position with respect to the first insertion hole 71, the engaging member 8 is in the most advanced position, and the housing 7 and the sheath 101 are engaged with each other via the engaging member 8. Therefore, when the operator pushes the pusher 2 to the innermost position with respect to the first insertion hole 71 and then releases a pressing force on the pusher 2, the pusher 2 returns to the retracted position shown in FIG. 1 due to the biasing member prepared for the pusher 2. In this state, the cartridge 1 is moved in a distal direction, and the clip unit 4 is taken out from the cartridge 1.

Subsequently, the clip unit 4 is inserted into a human body via a treatment tool channel of an endoscope and is brought approach to the tissue to be ligated by the arm member 3. Since the housing 7 is engaged with the sheath 101 by the engaging member 8, when the sheath 101 is rotated around the longitudinal axis C, the housing 7 also rotates, and an orientation of the arms 31 of the arm member 3 around the longitudinal axis C can be adjusted.

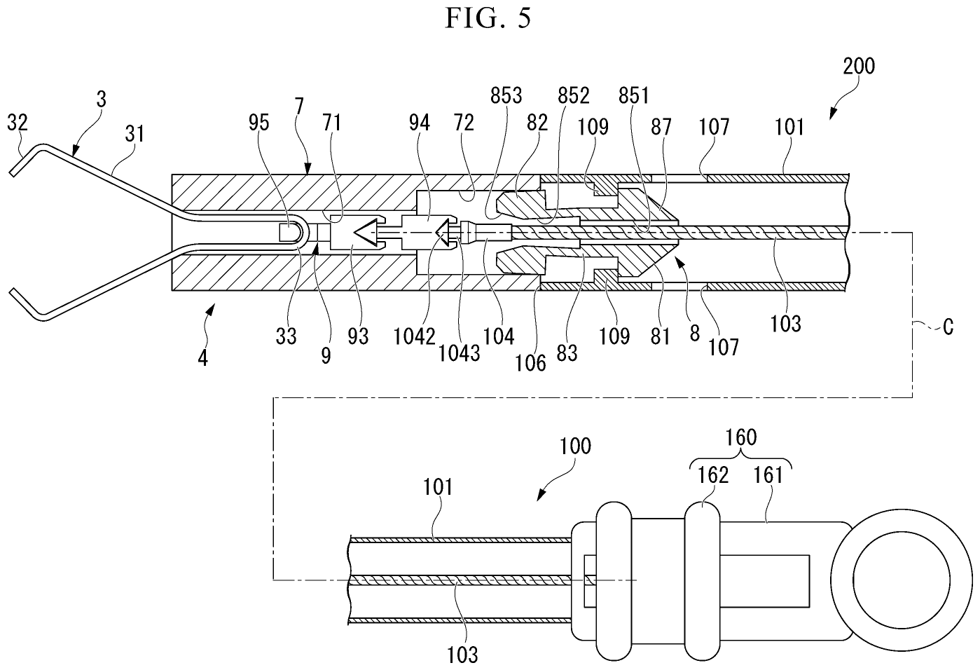

Then, when the slider 162 of the applicator 100 is advanced, the operation wire 103 advances with respect to the sheath 101 and the engaging member 8, as shown in FIG. 5. As the operation wire 103 advances, the arm member 3 advances with respect to the housing 7, and the pair of arms 31 protrudes from the second accommodation part 122 toward the distal side. Among the pair of arms 31, portions which protrude from the second accommodation part 122 toward the distal side is released from the pressing force by the second accommodation part 122 and open in a direction away from each other.

On the other hand, when the slider 162 is retracted with respect to the operation main body 161 and the operation wire 103 is retracted, the arm member 3 moves toward the proximal side. As a result, proximal parts of the pair of arms 31 are inserted into the housing 7, the pair of arms 31 move in a direction approaching each other, and then the arm member 3 is closed.

In the clip unit 4, since the sheath 101 and the housing 7 are engaged with each other via the engaging member 8, and the operation wire 103 is configured to be advanceable and retractable in this state, the arm member 3 is capable of advancing and retracting with respect to the housing 7. As a result, the tissue can be re-grasped by the arm member 3. That is, when the tissue is once sandwiched between the pair of claws 32 of the arm member 3 and then the arm member 3 is advanced relative to the housing 7, the pair of arms 31 is capable of being opened again, and a state in which the pair of claws 32 sandwich the tissue is capable of being released. When the arm member 3 is brought grasp to the tissue again with the pair of arms 31 being opened and is retracted relative to the housing 7, the tissue is capable of being sandwiched between the pair of claws 32 again.

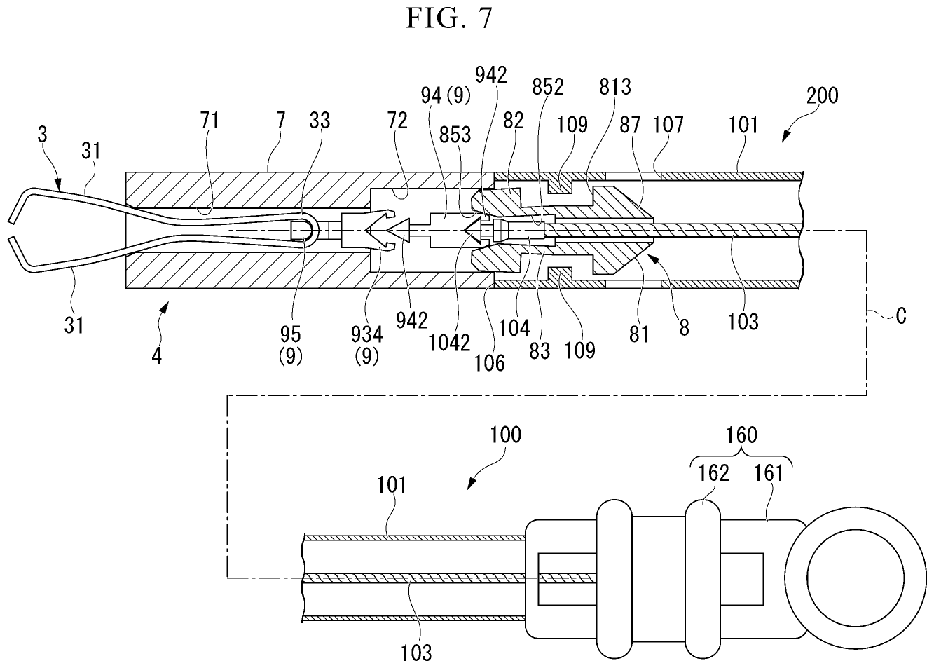

When the tissue is grasped by the arm member 3 in a desired state, the operator retracts the slider 162 to retract the operation wire 103 and the arm member 3. When the operation wire 103 and the arm member 3 retract while the tissue is grasped between the pair of claws 32, tension is applied to the connection body 92. As a result, as shown in FIG. 7, the pair of engaging arms 934 of the second connection body 93 are pushed and deformed by the conical part 922 in a direction away from each other, the conical part 922 falls off from the engaging hole 933, and the engagement between the first connection body 92 and the second connection body 93 is released.

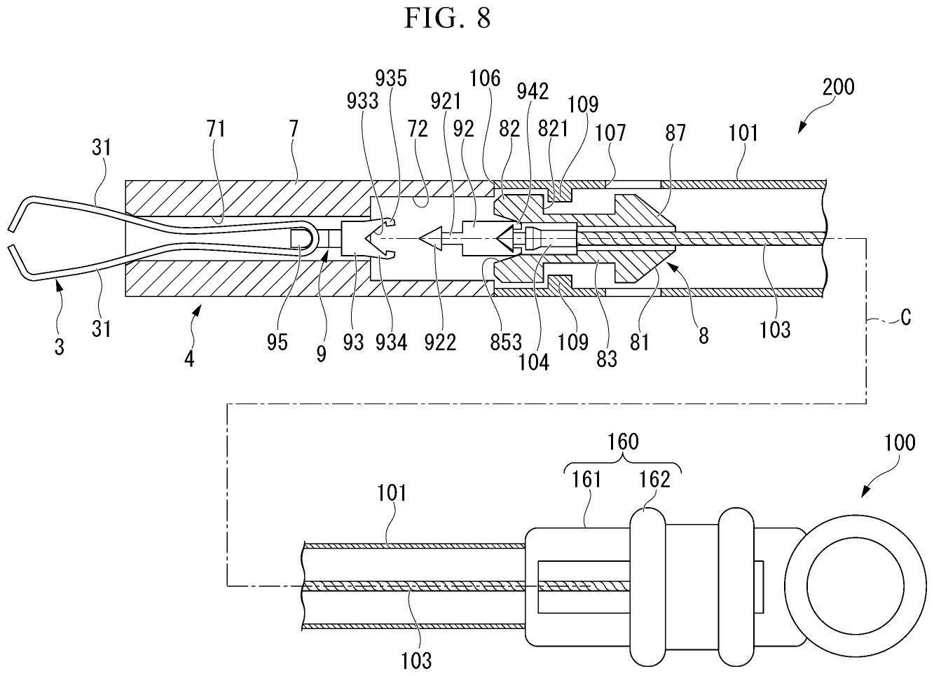

When the operation wire 103 is retracted until the engagement between the first connection body 92 and the second connection body 93 is released, the pair of engaging arms 942 of the first connection body 92 come into contact with the inner tapered surface 853 of the second insertion through hole 852. As a result, the pair of engaging arms 942 of the first connection body 92 are maintained in a state in which the engaged part 1042 is engaged, unlike the pair of engaging arms 934 of the second connection body 93. While the pair of engaging arms 942 are in contact with the inner tapered surface 853, a force is applied thereto in a retraction direction. Therefore, as the operation wire 103 retracts, a force is applied to the engaging member 8 in the proximal direction, and the engaging member 8 retracts with respect to the housing 7. When the engaging member 8 is pushed toward the proximal side by the first connection body 92, as shown in FIG. 8, the engaging member 8 comes off from the first insertion hole 71, and the engagement between the sheath 101 and the housing 7 by the engaging member 8 is released. That is, the disengaging operation between the first connection body 92 and the second connection body 93 and the operation of releasing the engagement between the sheath 101 and the housing 7 by the engaging member 8 are performed substantially at the same time. After the engagement between the housing 7 and the sheath 101 is released, the sheath 101 is retracted, and the tissue is ligated by the clip unit 4.

After the tissue is ligated by the first clip unit 4, the applicator 100 is removed from the body, and if necessary, a second clip unit 4 is loaded into the applicator 100 again. Specifically, after the applicator 100 is removed from the body, the operation wire 103 protrudes from the distal end 106 of the sheath 101, and the first connection body 92 connected to the connector 104 of the operation wire 103 is removed. Next, a second cartridge 1 in which the second clip unit 4 is accommodated is prepared. The operation wire 103 is retracted, the sheath 101 is inserted into the cartridge 1 in the same method as described above, and the cartridge 1 and the sheath 101 are engaged with each other via the engaging member 8. Thus, the clip unit 4 is loaded into the sheath 101. In this way, when a plurality of sites of tissue are ligated by a plurality of clip units 4, the clip units are capable of being continuously loaded without replacing the applicator 100.

According to the cartridge 1 according to the present embodiment, the clip unit 4 can be easily loaded into the applicator 100, and the clip unit 4 is repeatedly loaded into the applicator 100.

According to the cartridge 1 according to the present embodiment, the distal end part 105 of the sheath 101 is inserted into the insertion hole 15 of the main body 11, and the pusher 2 is movable in a direction crossing the main body 11. The pusher 2 is capable of pushing the engaging member 8 in an advance direction with respect to the housing 7. The engaging member 8 engages the sheath 101 with the housing 7 by the pusher 2 engaging the engaging member 8 with both the housing 7 and the sheath 101. As a result, the clip unit 4 is capable of being mounted in the applicator 100 by a simple operation in which the pusher 2 is pushed in a state in which the sheath 101 is inserted into the main body 11.

According to the cartridge 1 according to the present embodiment, the pusher 2 is configured to be movable in a direction crossing the longitudinal axis C of the elongated first insertion hole 71. An insertion direction of the sheath 101 into the cartridge 1 and a pushing direction of the engaging member 8 against the cartridge 1 are directions along the longitudinal axis C. On the other hand, the pusher 2 moves in the direction crossing the longitudinal axis C and pushes the engaging member 8. Therefore, the engaging member 8 is smoothly pushed in the direction of the longitudinal axis C by moving the pusher 2 in a direction different from a movement direction of the operation wire 103 while a state in which the sheath 101 is inserted into the main body 11 of the cartridge 1 is held. As a result, the sheath 101 is capable of being engaged with the housing 7 by pushing the pusher 2 and bringing the engaging member 8 to advance while maintaining the state that the cartridge 1 contacts with the distal end 106 of the sheath 101.

In the present embodiment, since the pusher 2 is configured to protrude into the insertion hole 15, the engaging member 8 is capable of being advanced in a state that the sheath 101 is inserted into the insertion hole 15.

According to the clip system 200 according to the present embodiment, the pusher 2 comes into contact with the engaging member 8 accommodated in the sheath 101 by passing through the through hole 107 formed in the outer circumferential surface of the sheath 101 and protruding into the first insertion hole 71. As a result, in a state in which the sheath 101 is inserted into the first insertion hole 71, the pusher 2 is configured to come into contact with the engaging member 8 disposed in the sheath 101, and the engaging member 8 is configured to be pushed by the pusher 2.

According to the clip system 200 according to the present embodiment, the inclined surface 21 is formed on the pusher 2, and the inclined surface 21 is configured to be able to come into contact with the engaging member 8. With such a configuration, the engaging member 8 is capable of being advanced while the pusher 2 is moved in a direction different from the advance direction of the engaging member 8.

In the present embodiment, an example in which the proximal end portion 81 of the engaging member 8 has the tapered portion 812, and the pusher 2 has the inclined surface 21, and the tapered portion 812 and the inclined surface 21 come into contact with each other is shown. However, the clip system is not limited to such a configuration example and may be configured to have an inclined surface on one of the pusher and the engaging member.

In the present embodiment, since the engaging member 8 is capable of protruding from the sheath 101 by the pusher 2, the cartridge 1 is capable of being mounted on the distal end 106 of the sheath 101, and the engaging member 8 is capable of protruding from the distal end 106 of the sheath 101. Therefore, as compared with a case in which the engaging member 8 located at the distal end 106 of the sheath 101 is pushed by the operation part 160 provided on the proximal end side of the applicator 100, the engaging member 8 is capable of being pushed with a simple structure.

In the present embodiment, an example in which the sheath 101 is capable of being inserted from the proximal side of the first insertion hole 71 in the direction of the longitudinal axis C is shown, but the insertion form of the sheath into the insertion hole is not limited thereto. For example, the sheath may be inserted into the insertion hole from the main body side of the cartridge.

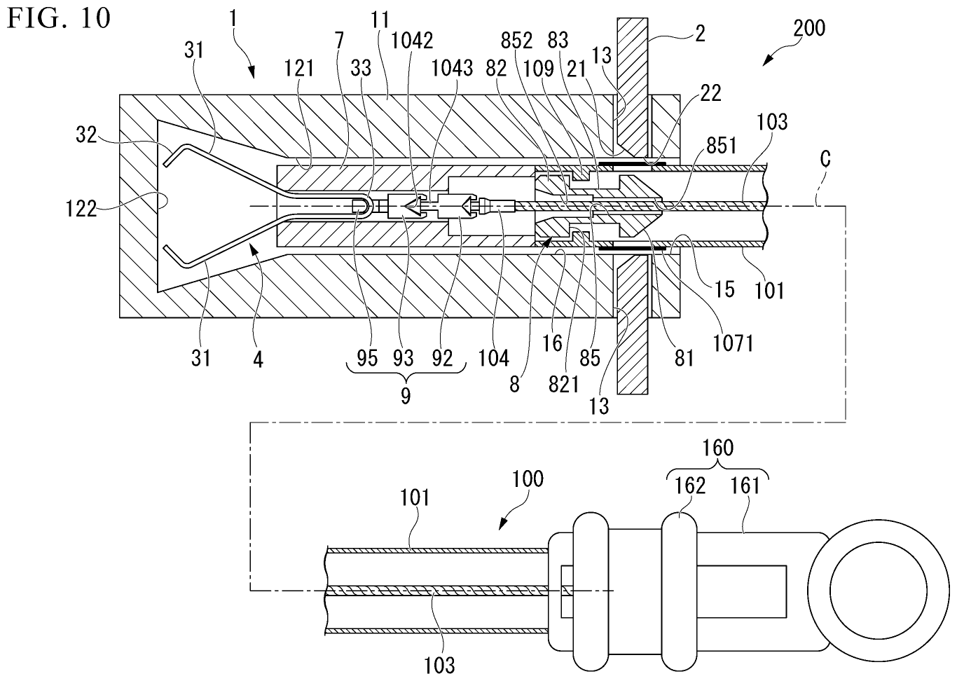

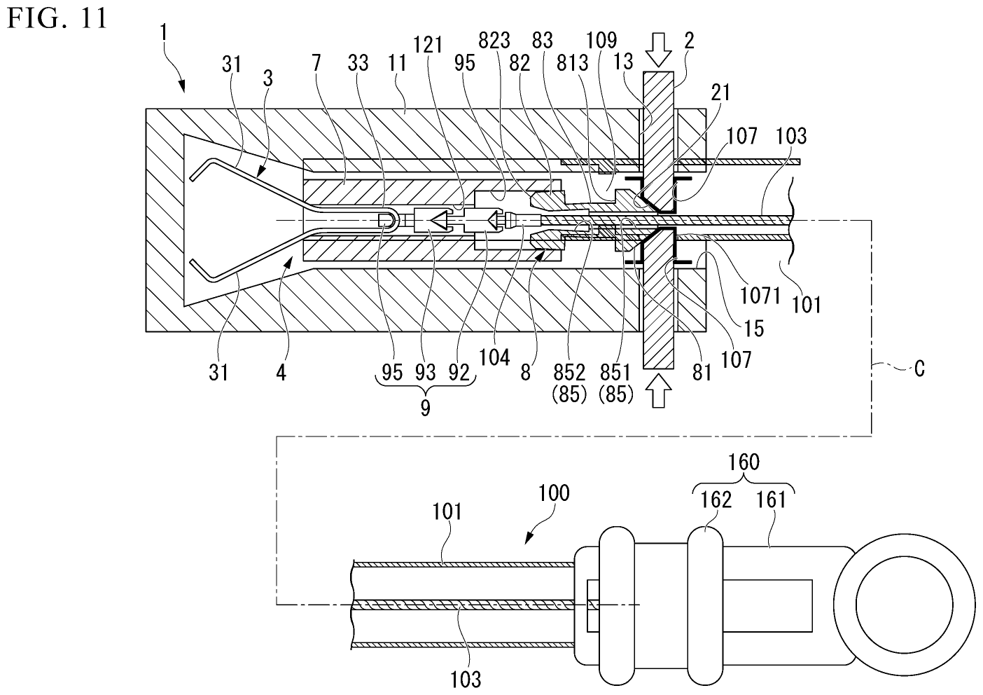

In the present embodiment, the example in which the pusher 2 protrudes into the first insertion hole 71 through the through hole 107 formed on the outer circumferential surface of the sheath 101 and comes into contact with the engaging member 8 accommodated in the sheath 101 is shown. However, the mode in which the pusher 2 protrudes into the first insertion hole 71 is not limited thereto. For example, as shown in FIGS. 10 and 11, the through hole 107 may be covered with an elastically deformable elastic film 1071, and the pusher 2 may protrude into the first insertion hole 71 by pressing the elastic film 1071 with the pusher 2 and deforming the elastic film 1071. In this case, the pusher 2 is capable of pushing the engaging member 8 with the elastic film 1071 interposed between the pusher 2 and the engaging member 8.

### Second Embodiment

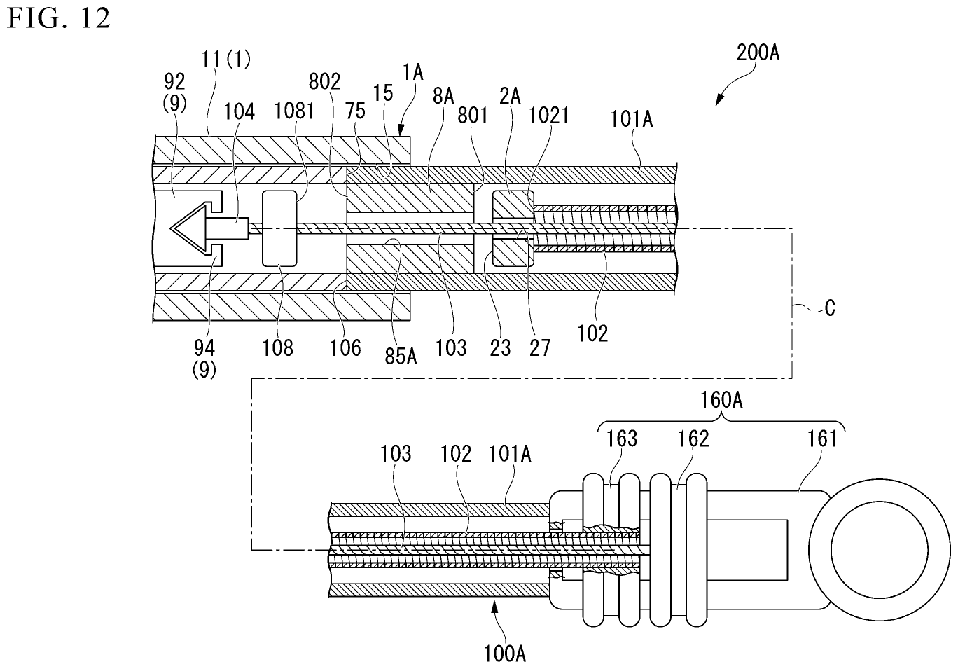

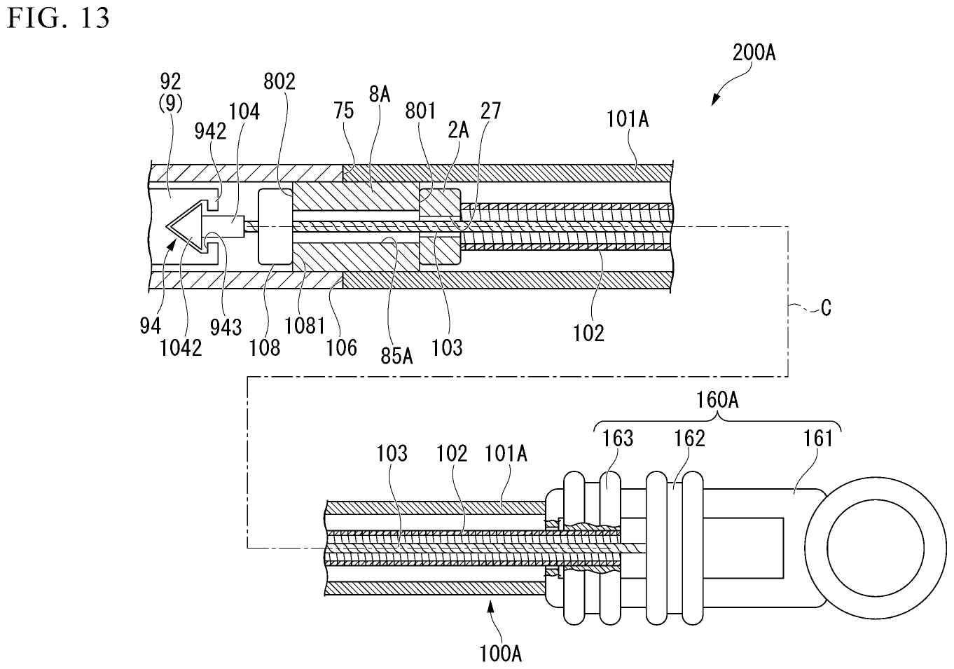

Next, a clip system 200A according to a second embodiment will be described with reference to FIGS. 12 and 13. In the following description, the same components as those already described in the first embodiment are designated by the same reference numerals, and duplicate description will be omitted.

In the present embodiment, the configuration in which the pusher pushes the engaging member against the housing 7 is different from that in the first embodiment.

The sheath 101 of the present embodiment is a coil sheath which does not have the convex part 109 and the through hole 107.

A pusher 2A is accommodated in the sheath 101. The pusher 2A of the present embodiment is provided at a distal end 1021 of an inner sheath 102. The inner sheath 102 is inserted into the sheath 101, and the operation wire 103 is inserted inside. The inner sheath 102 is disposed coaxially with the sheath 101 between the operation wire 103 and the sheath 101.

The inner sheath 102 is a coil sheath. A proximal end portion of the inner sheath 102 is fixed to a second slider 163 provided on the operation part 160A. The second slider 163 is capable of advancing and retracting with respect to the operation main body 161.

The pusher 2A is a substantially cylindrical member and is fixed to the distal end 1021 of the inner sheath 102. Therefore, the inner sheath 102 advances and retracts with respect to the sheath 101 by an advance and retraction operation of the second slider 163, and the pusher 2A advances and retracts as the inner sheath 102 advances and retracts. An insertion hole 27 which passes therethrough in the direction of the longitudinal axis C is formed in the pusher 2A. The pusher 2A is fixed to the inner sheath 102 by coaxially disposing central axes of the insertion hole 27 and a pipeline 1022 of the inner sheath 102.

An engaging member 8A of the present embodiment is made of a tubular member having a substantially cylindrical shape. The engaging member 8A is inserted into the conduit 111 of the distal end part 105 of the sheath 101. An outer circumferential surface of the engaging member 8A has an outer dimension which is capable of frictionally engaging with the inner surface of the sheath 101 and the inner surface of the first accommodation part 121. The engaging member 8A is capable of advancing and retracting in the conduit 111 and in the first accommodation part 121 when the engaging member 8A is pushed in the direction of the longitudinal axis C. The engaging member 8A frictionally engages with the inner surface of the sheath 101 and the first accommodation part 121. The engaging member 8A has an insertion through hole 85A which extends in the direction of the longitudinal axis C over the entire length thereof.

A distal pusher 108 is fixed to the operation wire 103. The distal pusher 108 is a distal end region of the operation wire 103 and is fixed to the proximal side further than the connector 104. The distal pusher 108 is a cylindrical member. The diameter of the distal pusher 108 is smaller than the inner diameter of the sheath 101 and the inner diameter of the first accommodation part 121 of the housing 7, and is larger than the inner diameter of an insertion through hole 85A of the engaging member 8A.

The operation wire 103 is inserted into the sheath 101 and the inner sheath 102, and a distal part of the operation wire 103 is inserted through the insertion through hole 85A of the engaging member 8A. The connector 104 and the distal pusher 108 are located on a distal side further than the engaging member 8A.

A cartridge 1A does not include the pusher 2 and the slide hole 13 of the first embodiment, and other configurations are the same as those of the first embodiment.

Next, an operation and an engagement method of the clip system 200A according to the present embodiment will be described.

The distal end 106 of the sheath 101 is inserted into the insertion hole 15 of the cartridge 1A in which the clip unit 4 is accommodated, and is brought into contact with the proximal end 75 of the housing 7. As in the first embodiment, the operation wire 103 is advanced, the engaged part 1042 is pushed to the engaging hole 941 side of the clip unit 4, and the connector 104 is engaged with the connection member 9.

Next, the second slider 163 is advanced with respect to the operation main body 161, and the inner sheath 102 is advanced with respect to the sheath 101. After the distal end 23 of the pusher 2A comes into contact with the proximal end 801 of the engaging member 8A, the second slider 163 is further advanced. As a result, the pusher 2A pushes the engaging member 8A in the distal direction, and the engaging member 8A advances and protrudes toward the distal side further than the distal end 106 of the sheath 101. As shown in FIG. 13, the engaging member 8A is pushed toward the distal side by the pusher 2A until the engaging member 8A is inserted into the first insertion hole 71 of the housing 7 and comes into contact with the proximal end 1081 of the distal pusher 108. As a result, the distal end 106 of the sheath 101 and the housing 7 are engaged with each other via the engaging member 8A.

The cartridge 1A is removed from the clip unit 4, and the installation of the clip unit 4 to the sheath 101 is completed. Then, the distal end part 105 of the sheath 101 is inserted into the body in the same method as in the first embodiment, and the target tissue is ligated by the clip unit 4.

After the target tissue is ligated by the clip unit 4, the engagement between the housing 7 and the sheath 101 is released. The slider 162 of the operation part 160 is slid toward the proximal side. The pair of engaging arms 934 of the second connection body 93 are pushed and deformed by the conical part 922 in the direction away from each other, and the conical part 922 falls off from the engaging hole 933. Similar to the first embodiment, the engagement between the first connection body 92 and the second connection body 93 is released. At the same time, the distal pusher 108 pushes the engaging member 8A toward the proximal side, the engaging member 8A moves toward the proximal side, and engagement between the housing 7 and the sheath 101 by the engaging member 8A is released. As a result, the tissue is ligated by the clip unit 4.

According to the clip system 200A according to the present embodiment, similar to the first embodiment, the clip unit 4 is easily loaded into the applicator 100, and the clip unit is capable of being repeatedly loaded into the applicator 100.

According to the clip system 200A according to the present embodiment, the pusher 2A is configured to be in contact with the proximal end 801 of the engaging member 8A by advancing and retracting with respect to the operation wire 103, and the engaging member 8A is configured to be press-fitted into the first accommodation part 121 of the housing 7. As a result, the housing 7 and the sheath 101 is capable of being engaged with each other by advancing and retracting the engaging member 8A in the sheath 101. Therefore, the housing 7 and the sheath 101 is capable of being engaged with each other with a simple configuration.

In the present embodiment, since the operation wire 103 has the distal pusher 108, the engaging member 8A is capable of being pushed and moved toward the proximal side by retracting the operation wire 103. Therefore, the engaging member 8A is capable of advancing and retracting with respect to the sheath 101 and the housing 7. As a result, the clip unit 4 is removably mounted on the sheath 101. Further, the clip unit 4 is repeatedly loaded into the applicator 100.

### Third Embodiment

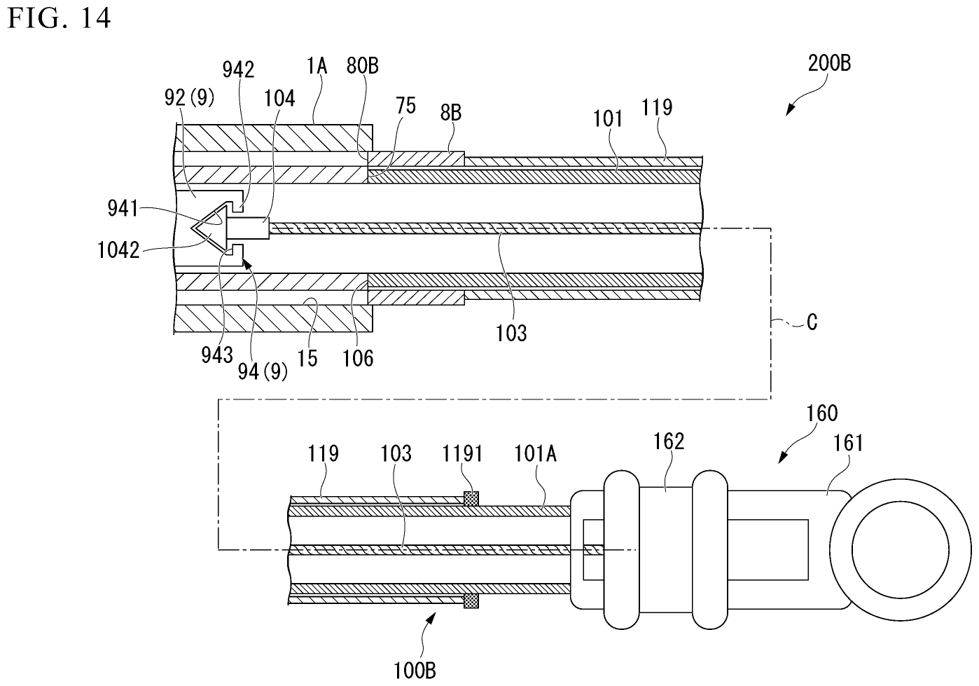

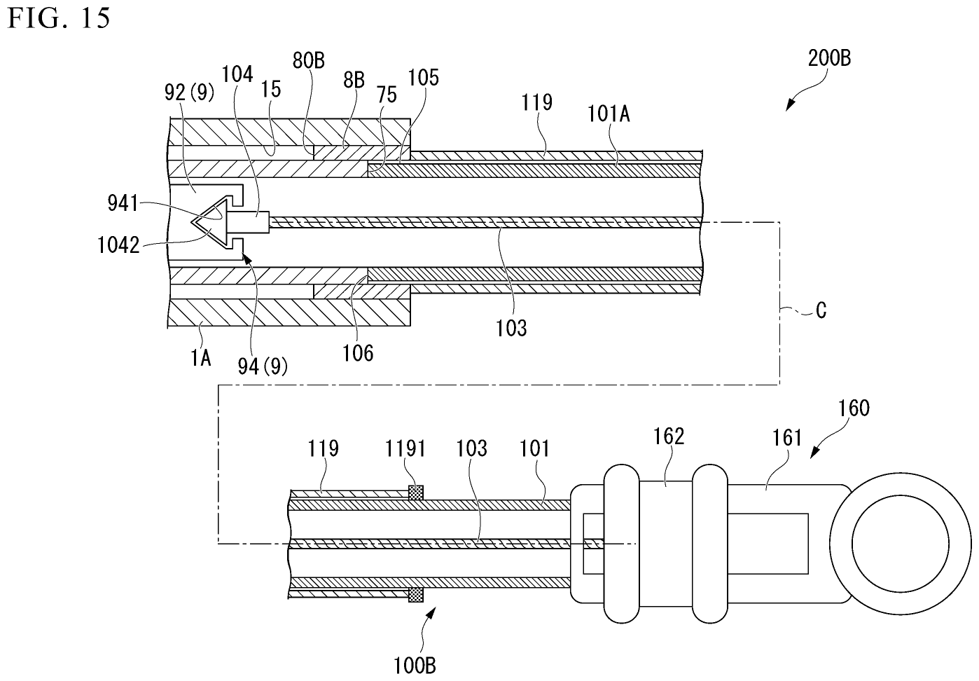

Next, a clip system 200A according to a third embodiment will be described with reference to FIGS. 14 and 15.

In the present embodiment, the configuration of the engaging member and the configuration in which the pusher pushes the engaging member against the housing 7 are different from those of the first embodiment.

As shown in FIG. 14, an applicator 100B according to the present embodiment includes an outer sheath 119. The outer sheath 119 serves as a pusher for the engaging member 8B. The outer sheath 119 is disposed coaxially with the sheath 101, and the sheath 101 into which the operation wire 103 is inserted is inserted therein. A grip part 1191 is provided at a proximal end of the outer sheath 119. The outer sheath 119 is provided to be advanceable and retractable in the direction of the longitudinal axis C with respect to the sheath 101.

The outer sheath 119 includes an engaging member 8B. The engaging member 8B is fixed to a distal end of the outer sheath 119. The engaging member 8B is a tubular member into which the sheath 101 and the housing 7 is capable of being inserted. The engaging member 8B is, for example, a tube made of a soft resin or a coil of which a diameter is capable of being reduced. The engaging member 8B is provided to be advanceable and retractable with respect to the sheath 101 and the housing 7 while being in contact with the outer circumferential surfaces of the sheath 101 and the housing 7.

Since the engaging member 8B is fixed to the distal end of the outer sheath 119, the engaging member 8B advances in the distal direction toward the housing 7 due to the advance of the outer sheath 119. The engaging member 8B is configured so that the housing 7 and the sheath 101 is capable of being engaged with each other by friction between the outer circumferential surface of the housing 7 and the outer circumferential surface of the sheath 101.

Similar to the second embodiment, the sheath 101 of the present embodiment is a coil sheath which does not have the convex part 109 and the through hole 107.

In a clip system 200B according to the present embodiment, the same cartridge 1A as in the second embodiment is used.

Next, an operation and an engagement method of the clip system 200B according to the present embodiment will be described.

The distal end 106 of the sheath 101 is inserted into the insertion hole 15 of the cartridge 1A in which the clip unit 4 is accommodated, and is brought into contact with the proximal end 75 of the housing 7. As in the first embodiment, the operation wire 103 is advanced to push the engaged part 1042 toward the engaging hole 941 side of the clip unit 4, and the connector 104 is engaged with the connection member 9.

Next, the operator grips the grip part 1191 and advances the outer sheath 119 with respect to the sheath 101. As the outer sheath 119 advances, the engaging member 8B advances and protrudes toward the distal side further than the distal end 106 of the sheath 101, and the distal end 802 of the engaging member 8B enters the insertion hole 15 of the cartridge 1A. When the outer sheath 119 is further advanced, the distal end 106 of the sheath 101 and the proximal end 75 of the housing 7 are inserted into the engaging member 8B, as shown in FIG. 15. An inner circumferential surface of the engaging member 8B comes into contact with the outer circumferential surface of the sheath 101 and the housing 7, and the distal end 106 of the sheath 101 and the proximal end 75 of the housing 7 are engaged with each other via the engaging member 8B.

Subsequently, the cartridge 1A is removed from the clip unit 4, and the installation of the clip unit 4 to the sheath 101 is completed. Then, the distal end part 105 of the sheath 101 is inserted into the body in the same method as in the first embodiment, and the target tissue is ligated by the clip unit 4.

After the target tissue is ligated by the clip unit 4, the engagement between the housing 7 and the sheath 101 is released. The grip part 1191 of the outer sheath 119 is slid toward the proximal side. When the grip part 1191 is pulled toward the proximal side, the engaging member 8B moves toward the proximal side, and the engagement between the housing 7 and the sheath 101 by the engaging member 8B is released. After that, when the operator pulls the slider 162 toward the proximal side, the pair of engaging arms 934 of the second connection body 93 is pushed and deformed by the conical part 922 in the direction away from each other, and the conical part 922 falls off from the engaging hole 933. Similar to the first embodiment, the engagement between the first connection body 92 and the second connection body 93 is released. As a result, the tissue is ligated by the clip unit 4. The releasing of the engagement between the housing 7 and the sheath 101 by the engaging member 8B may be performed after an operation in which the engagement between the first connection body 92 and the second connection body 93 is released is performed.

According to the clip system 200B according to the present embodiment, Similar to the first embodiment, the clip unit 4 is easily loaded into the applicator 100B, and the clip unit 4 is capable of being repeatedly loaded into the applicator 100B.

According to the clip system 200B according to the present embodiment, the engaging member 8B is fixed to the distal end of the outer sheath 119, and the engaging member 8B is configured to be advanceable and retractable by advancing and retracting the outer sheath 119 with respect to the sheath 101. As a result, the housing 7 and the sheath 101 is capable of being engaged with each other by advancing the engaging member 8B on the outside of the sheath 101. Therefore, the housing 7 and the sheath 101 is capable of being engaged with each other with a simple configuration.

In the present embodiment, since the outer sheath 119 includes the engaging member 8B, the engaging member 8B is moved toward the proximal side by retracting the outer sheath 119. Therefore, the engaging member 8B is capable of advancing and retracting with respect to the sheath 101 and the housing 7. As a result, the clip unit 4 is removably mounted on the sheath 101, and the clip unit 4 is capable of being repeatedly loaded into the applicator 100B.

Although the embodiments of the present invention have been described in detail with reference to the drawings, the specific configuration is not limited to these embodiments, and design changes and the like within a range not deviating from the gist of the present invention are also included.

In addition, the components shown in each of the above-described embodiments and modified examples can be appropriately combined and configured.

What is claimed is:

1. A cartridge configured to accommodate a clip unit that includes an arm member and a tubular housing into which the arm member is inserted, the cartridge further configured so that the clip unit is able to be mounted in an applicator, the applicator having a sheath that is engageable with the housing and having an operation wire which is engageable with the arm member, the cartridge comprising:
a main body having an insertion hole, the insertion hole being configured to receive the sheath; and
a pusher configured to be movable with respect to the main body, the pusher configured to be able to come into contact with an engaging member by moving the pusher with respect to the main body in a state in which the sheath is inserted into the insertion hole, the pusher further configured to push the engaging member against the housing while in contact with the engaging member, and to engage the sheath with the housing by engaging the engaging member with both the housing and the sheath, the engaging member being provided in the sheath.

2. The cartridge according to claim 1, wherein the insertion hole has an elongated shape into which the sheath is able to be mounted, and
the pusher is configured to be movable in a direction crossing a longitudinal axis of the insertion hole.

3. The cartridge according to claim 2, wherein the pusher is configured to protrude into the insertion hole.

4. The cartridge according to claim 3, wherein the pusher is configured to come into contact with the engaging member, the engaging member being accommodated in the sheath, by passing through an opening formed in an outer circumferential surface of the sheath and protruding into the insertion hole.

5. The cartridge according to claim 4, wherein the pusher has an inclined surface that is inclined with respect to the longitudinal axis of the insertion hole, and
the inclined surface of the pusher is configured to be able to come into contact with the engaging member.

6. The cartridge according to claim 5, wherein the inclined surface includes a chamfered corner portion on a distal side of the inclined surface.

7. A clip system comprising:
a clip unit including an arm member and a housing having a tubular shape, the arm member being inserted into the housing;
an applicator having a sheath that is engageable with the housing and an operation wire that is engageable with the arm member; and
an engaging member that is provided in the applicator and configured to be engaged with both the sheath and the housing and configured to move from the sheath toward the housing.

8. The clip system according to claim 7, further comprising a pusher configured to move the engaging member from the sheath toward the housing, the pusher moving with respect to the sheath, the engaging member engaging with both the sheath and the housing as a consequence of the movement by the pusher.

9. The clip system according to claim 8, wherein the pusher is further configured to:
come into contact with the engaging member by moving with respect to the sheath, and
push the engaging member against the housing, and
wherein the pusher is configured to press-fit the engaging member into the housing.

10. The clip system according to claim 8, wherein the pusher is further configured to:
come into contact with the engaging member by moving with respect to the sheath, and
push the engaging member against the housing, and
wherein the pusher is configured to allow the engaging member to protrude from the sheath.

11. The clip system according to claim 8, wherein an opening is formed in an outer circumferential surface of the sheath, and
wherein the pusher is configured to:
come into contact with the engaging member by moving with respect to the sheath,
push the engaging member against the housing, and
come into contact with the engaging member, the engaging member accommodated in the sheath, by passing through the opening of the sheath and protruding into the insertion hole.

12. The clip system according to claim 8, further comprising a cartridge including a main body having an accommodation part into which the clip unit is accommodated, wherein:
the main body includes an insertion hole into which the sheath is inserted,
the pusher is configured to:
come into contact with the engaging member by moving with respect to the sheath and
push the engaging member against the housing, and
the pusher is provided on the cartridge and is movable with respect to the main body.

13. The clip system according to claim 8, wherein the engaging member is accommodated in the sheath,
the pusher is configured to:
come into contact with the engaging member by moving with respect to the sheath and
push the engaging member against the housing,
the pusher is accommodated in the sheath, and
the pusher is configured to be able to come into contact with the engaging member by advancing and retracting with respect to the operation wire.

14. The clip system according to claim 8, wherein the pusher comprises the sheath and an outer sheath into which the housing is insertable,
the engaging member is provided on the outer sheath, and
the engaging member is able to engage the housing with the sheath due to friction between an outer circumferential surface of the housing and an outer circumferential surface of the sheath as a consequence of the outer sheath moving the engaging member toward the housing.

15. A method of mounting a clip, comprising:
approaching a sheath to a cartridge that accommodates an arm member and a housing into which the arm member is inserted;
moving an engaging member at least partially accommodated in the sheath from the sheath toward the housing in a state in which the sheath is within a predetermined distance from the housing; and
moving the engaging member so as to engage the engaging member with both the housing and the sheath.

16. The method according to claim 15, further comprising inserting the engaging member into the housing by moving the engaging member from the sheath toward the housing.

17. The method according to claim 15, further comprising closing the arm member by moving the arm member from the housing toward the sheath.

18. The method according to claim 15, wherein the engaging member is moved from the sheath toward the housing by a pusher configured to move independently of the operation wire.

19. The method according to claim 18, wherein, when the engaging member is moved, the pusher is brought into contact with the engaging member by moving the pusher with respect to the cartridge in a state in which the sheath is inserted into an insertion hole provided in the cartridge, and
the engaging member is pushed against the housing by moving the pusher from a state in which the pusher is in contact with the engaging member.
